(19) United States
(12) Patent Application Publication
(10) Pub. No.: US 2009/0005669 A1
(43) Pub. Date: Jan. 1, 2009

(54) System and method for performing remote radiological examinations

(75) Inventors: Sebastian Schmidt, Weisendorf (DE); Sabine Schaeffer-Kundler, Herzogenaurach (DE)

Correspondence Address: BRINKS HOFER GILSON & LIONE, P.O. BOX 10395, CHICAGO, IL, 60610, US

(73) Assignee: Siemens Aktiengellsellschaft

(21) Appl. No.: 11/824,003

(22) Filed: Jun. 28, 2007

(51) Int. Cl. A61B 6/00

(52) U.S. Cl. 600/407

(57) ABSTRACT

A system and method is disclosed for enabling the planning and conduct of patient examinations by an imaging modality, where a remotely located technician plans and conducts at least part of the examination protocol. A local facility may have an imaging modality such as magnetic resonance imaging (MRI) equipment, and a portion of the preparation for, and conduct of, the data acquisition may be performed by a local technician. The remotely located technician may control one or more of the data acquisition parameters, the timing of the administering of contrast agents, and the modification of values of data acquisition parameters during the course of the image data acquisition by the imaging modality.

### TECHNICAL FIELD

The present application relates to a system and method for performing radiological examinations.

### BACKGROUND

In some countries or geographical portions thereof, there are shortages of qualified personnel for performing radiological examinations. This may lead to high salary costs and under-utilization of expensive diagnostic equipment, particularly when work shifts cannot adequately be staffed. This is a significant problem when using imaging modalities that require a high level of qualification or training to properly configure the equipment and to interpret the results, such as Magnetic Resonance Imaging (MRI) or computed tomography (CT). In other areas, countries, or times of day, the availability of appropriately trained personnel may be much better.

Another problem is that some types of imaging studies are not performed very often at a single facility. Particularly at smaller sites or medical institutions, having a limited number of patients, it is not efficient to train and maintain proficiency of personnel so as to be able to perform procedures that are infrequently performed, such as only a few times a month or a year. Such lack of personnel may also limit the availability of medically desired tests and studies which can be performed at other locations.

Outsourcing of reading and report writing for radiological examinations is known and is being used more frequently to partially mitigate this problem. However, for the examination itself, the only adopted solution has been to reduce the complexity of the examination and enhance the usability of the equipment, so that personnel having lesser qualifications can perform an examination at the local facility. But, this approach negates some of the benefits of newly developed examinations and equipment, or examinations which have a high intrinsic complexity. This may include examinations where the course of the examination is dependent on evaluation of images made during the examination itself, and for which the time lag in outsourcing the reading of the images is unacceptable, such as in cardiological examinations.

### SUMMARY

A system and method for performing medical imaging is described. The system includes a local facility and a remote facility. The local facility may include an imaging modality, a local imaging modality control interface, and a communications interface. The remote facility includes a computer, a image display unit and a communications interface.

In an aspect, the local facility may be a medical imaging facility, including an imaging modality, a imaging modality control interface, and a communications interface. The imaging modality control interface may be configured to interpret data received by the communications interface so as to establish or modify parametric values associated with the operation of the imaging modality.

In another aspect, an imaging modality control facility includes a computer having an operator interface, an image display unit, and a communications interface, where the computer may be configured to interpret data received by the communications interface so as to display an image of data obtained by an imaging modality, and to display and control parametric values of operation of a remotely located imaging modality.

A method of medical imaging, includes providing an imaging modality and interfacing the imaging modality with a communications interface having an interface to a communications network; sending information including image data and parametric settings of the imaging modality to the communications interface for transmission on the communications network; and, receiving information from the communications interface causing a change to one or more of the parametric settings of the imaging modality.

In another aspect, a method of performing medical imaging includes providing a computer configured to accept data including medical image data and imaging modality parametric control data received by a network communications interface; displaying at least one of a medical image or imaging modality parametric control data on a image display unit; and changing a value of at least one of the imaging modality parametric control data and transmitting the data including at least the changed control value using the network communications interface.

In yet another aspect, computer-readable medium is described, the contents of which enable a computer system to perform a method of accepting remote control of an imaging modality, including storing information characterizing the parameters of operation of an imaging modality; transmitting the parameters of operation to a network interface so as to be communicated to a remote location; receiving data from the remote location resulting in a change in a value a parameter of operation; controlling the operation of the imaging modality in accordance with at least the changed value; and transmitting image data obtained by the imaging modality to the network interface so as to be communicated to a remote location.

In still another aspect, a computer-readable medium is described, the contents of which enable a computer system to perform a method of remote control of an imaging modality, including receiving information characterizing parameters of operation of an imaging modality over a network interface; modifying a value of at least one of the parameters of operation; transmitting the modified value over a network interface to a remote location having the imaging modality; and receiving data obtained by the imaging modality based on the at least one modified value.

### BRIEF DESCRIPTION OF THE DRAWINGS

FIG. 1 is a block diagram of a local imaging facility and a remote facility; and;

FIG. 2 is a block diagram illustrating the steps in a method where a technician at a remote facility provides planning or control of an imaging procedure.

### DETAILED DESCRIPTION

Exemplary embodiments may be better understood with reference to the drawings, but these examples are not intended to be of a limiting nature. Like numbered elements in the same or different drawings perform equivalent functions.

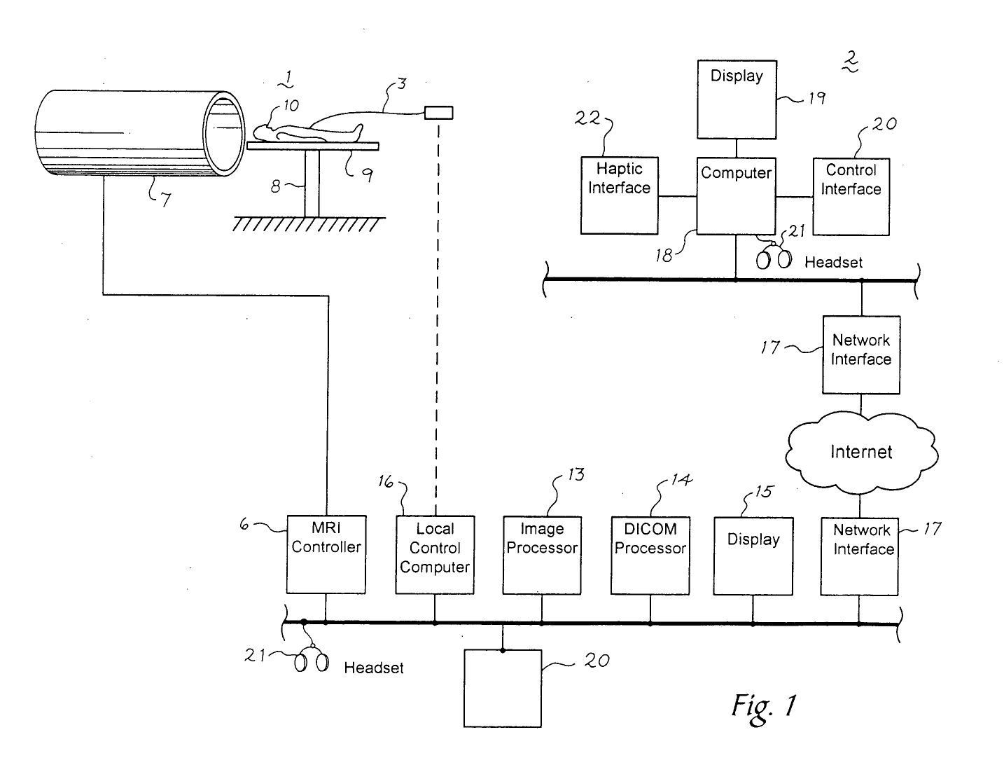

A system and method is described for performing remote radiological examinations. As shown in FIG. 1, the system comprises a local facility 1, which may be a stand-alone MRI center, or a similar facility in a hospital, and a remote facility 2, which may be located either in the local area or at a distance, and be connected to the local facility 1 by a suitable data and voice communications means such as the Internet, a wide area network, a local area network, a wireless network, or any combination thereof, so as to effect data and voice communications, including the use of digitized voice protocols such as Voice over Internet Protocol (VoIP).

The local facility 1 may include a magnetic resonance imaging (MRI) or computed tomography (CT) imaging modality 7, or the like, and have a patient examination table 9 mounted to a fixture 8, a robot, or the like, for the purpose of introducing a patient 10 into the imaging modality 7, and positioning the patient 10 for examination. The imaging modality 7, which may be an MRI device, may have ancillary equipment such as gradient coils and receiving coils (not shown), and be connected to a controller 6, which may generate gradient magnetic fields, radio frequency (RF) fields, and the like, in response to parameter settings received from a local control interface 16. Such a local control interface 16 may also include a keyboard, a computer, which may act as an interface between the local operator and the remainder of the system, and a display 15, which may serve to display locally rendered images as obtained by the imaging modality 7, as well as control and communications information and data.

Each type of imaging modality has a particular suite of hardware, and software programs that may execute on an image processor 13, so as to convert the data obtained by the imaging modality 7 into viewable images. This processing may be performed as the data is acquired (so called “real-time”) or at a later time. The image processing software may use various known or to be-developed algorithms to produce images of planes or slices of the patient, volume renderings, segmentations, and the like, the characteristics thereof depending on the imaging modality selected and the operating parameters thereof. The data and the images may be archived, or distributed using any technique, however it is common today to use a protocol known as DICOM (Digital Communications in Medicine). The various devices in the local examination facility that exchange digital data and control may be linked together by a local area network (LAN), which may use, for example, the Ethernet protocol. Point-to-point connections may also be used, for example, for the magnetic gradient control and for the pulsed RF energy produced by the MRI controller 6.

Equipment for administering contrast agents by intravenous means 3, and other monitoring equipment such as an electrocardiogram (EKG) may be provided.

The equipment may interface with a network interface 17, which may be considered to be a server or router to connect the local facility 1 with the remote facility 2 using a communications network. The network interface 17 may connect to any one or more of, for example, a facility-wide local area network, a campus-wide network, the Internet, or the like.

The remote facility 2 may be located at a commercial establishment such as a service provider. Equally, however, the remote facility 2 may be located at the medical facility or at another medical facility providing service to one or more local facilities 1.

The remote facility 2 has a computer 18, with a control interface 20, and may have a manipulator 22 for the situation where the use of haptic controls for remote manipulation of equipment is desired. A display 19 provides for the display of image and other data transmitted from the local facility 1 to the remote facility 2, and for the display and control of imaging modality parameters. An audio interface 21, may be a telephone handset, a cellular telephone, a headset, or the like, so that a person at the remote facility 2 may engage in audio communications with another person at the local facility 1. A speaker and pick-up microphone (not shown) may provide for what may be termed an intercom capability so that the remote technician may communicate with the local technician or the patient 10.

Visualization techniques of medical images have been developed with the goal of providing a better comprehension of the image data obtained by one or more imaging modalities. User interaction during the visualization process permits the user to change parameters and obtain a dynamic navigation process. The entire image data base for the examination, obtained for example by an MRI, or by a CT or CT-like device, or the like, may be retained at the local examination site 1, and selected images or portions thereof transmitted to the service provider at the remote location 2. The service provider display and computer may be used to visualize the data.

A goal of visualization is to show an interior portion of the patient body, such as the heart, to allow the identification of its inner regions and structures. In general, visualization may be a projection process of a multidimensional data set in a plane. As such, the physician may wish to view the patient from another vantage point. When using a CT imaging modality, the expert may control the rendering of the image reconstructed from a data base of the image data. Cross-sections oriented at various cut planes may be used to show sections of the patient body in different orientations from that used in the original image processing. The effect is another exam without having to submit the patient to another tomography scan having an additional X-ray dose.

However, in the MRI imaging modality, a variety of the parameters governing the examination may have been established in the examination planning process, and may be adjusted during the examination. These may lead to a data set which is not optimum for the specific study, and additional data may not have been stored to permit overcoming the limitation of the initial examination. That is, adjustment of the examination parameters after the data is obtained is limited. For example, MRI images having the description “Axial head, 22 cm FOV, 5 mm Thk, SE, TR/TE=5500/165 ms, 2 Nex, 512×256 matrix” and “Axial head, 22 cm FOV, 5 mm Thk, SE, TR/TE=450/14 ms, 1 Nex, 256×192 matrix”, may differ substantially in appearance and utility, even though the images are of the same body section.

MRI technology continues to evolve and thus the MRI exam may be performed on equipment whose technical characteristics may vary from the optimum “state-of-the-art” equipment that might have been used. In such cases, the closest approximation to the desired characteristics may be obtained by judicious choice of the type of pulse sequence to be used, such as spin-echo, inversion recovery and gradient recalled echo and other examination parameters. Generally the selection of pulse sequence, repetition time, gradient characteristics, and the like, are made with a view towards optimizing the contrast between the received signals for differing tissue types, and enhancing the visibility of tissue which may be related to a pathology or syndrome.

In another aspect, the MRI scan is often particularized to a specific principal plane, which may be the axial, coronal, or sagital plane, in body-oriented coordinates. However, other planes may be selected, and then the gradient magnetic fields may be adjusted so as to obtain data from a plane oriented at an angle to the principal planes. Since the image plane is defined by magnetic gradients, and the examination time for MRI is currently longer than for CT scans, a complete set of data needed to subsequently reconstruct a plane other than the plane selected for the examination may not now be routinely obtained. Further, the length of time which may be needed to obtain the data requires consideration of the types of pulse sequences used so as to keep the specific absorption rate (SAR) of the radio frequency and magnetic energy within safety bounds. This is also a consideration for the acoustic exposure as, presently, the changes in magnetic fields are associated with material distortions in the equipment that produce loud impulsive noises.

In the MRI technique, image artifacts may be produced by equipment malfunctions, and are preferably identified during the examination so that they may be rectified by a local technician or a service technician. However, there are other image artifacts that may be mitigated by proper planning, parameter selection, and instrumentation. Where images of blood vessels or the heart are concerned, the MRI data acquisition may be gated so as to occur at a particular portion of the cardiac cycle so as to, for example, mitigate the effects of motion, or to use motion to differentiate flowing blood from other tissue. Where an EKG is used for synchronization, the MRI data may be acquired at a specified delay time, for example, with respect to the R-wave. Other sensors may be used for synchronization, such as a respiratory sensor.

Where blood flow studies are being conducted, a number of different pulse sequences may be selectable to perform time-of-flight angiography, phase-contrast angiography or contrast-enhanced angiography. In contrast-enhanced sensing, a paramagnetic contrast agent, typically containing Gadolinuum (Gd) metal, is administered during the examination.

Other image parameters are used to identify different tissue types in the same image slice, such as fat as compared with water, or tumors, and the specific pulse sequences and other image parameters are specifically selected in accordance with the diagnostic objective of using the MRI imaging modality.

Moreover, the technician may have to select the appropriate MRI receiving coils from a variety of generic types such as surface, volume and internal, each of which has a number of variants.

Instructions for configuring computing devices to perform the operations and methods disclosed herein may be provided on computer-readable storage media or memories, such as a cache, buffer, RAM, ROM, flash memory, removable memory media, a hard drive or other computer readable storage media. Computer readable storage media include various types of volatile and nonvolatile storage media. The functions, acts or tasks illustrated in the figures or described herein may be executed in response to one or more sets of instructions stored in or on computer readable storage media. The functions, acts or tasks are independent of the particular type of instruction set, storage media, processor or processing strategy and may be performed by software, hardware, integrated circuits, firmware, micro code and the like, operating alone or in combination. Likewise, processing strategies may include multiprocessing, multitasking, parallel processing and the like.

The instructions may be stored on a removable media device for reading by local or remote systems. Alternatively, the instructions may be stored in a remote location for transfer through a computer network, a local or wide area network, or over telephone lines. In yet other embodiments, the instructions are stored within a given computer or system.

As there may be a multiplicity of users situated at geographically distributed locations, a web-based architecture may be used. Where the term “web” or “Internet” is used, the intent is to describe an internetworking environment, including both local and wide area networks, where defined information transmission protocols are used to facilitate communications between diverse, possibly geographically dispersed, entities. An example of such an environment is the world-wide-web (WWW) and the use of the TCP/IP data packet protocol, and the use of Ethernet or other hardware and software protocols for some of the data paths. Wireless communications may be used for all or a portion of the communications infrastructure.

The web infrastructure may include a plurality of computers and ancillary equipment performing functions such as a router or a server, data base storage, data transmission and reception over dedicated or switched circuits, and is generally provided by others as a service.

Wireless communication means may include, audio, radio, lightwave or other technique not requiring a physical connection between a transmitting device and a corresponding receiving device. While the communication may be described as being from a transmitter to a receiver, this does not exclude the reverse path, and a wireless communications device may include both transmitting and receiving functions.

An example of the method of the remote performance of examinations using an imaging modality, such as an MRI, may be the following scenario:

When any of the previously described situations makes the services of a non-co-located radiological technician desirable or necessary in the planning and conduct of a radiological examination; the services of the remotely located technician may be engaged, on a per examination basis, on a contract basis for a fixed number of examinations, or as an employee providing the service for multiple examination rooms, either in a local facility or at geographically dispersed facilities.

The patient is brought into the examination room and an EKG or other patient monitoring equipment is connected. The patient is placed on an examination table and an intravenous (IV) needle placed in the patient if a contrast agent is to be used in the examination. A contrast agent injector is then attached to the needle. This procedure may be performed either in the MRI equipment room or at a nursing station located nearby.

The examination table is brought to the vicinity of the MRI sensor and either a local medical technician or a physician may then place the receiving coils for a magnetic resonance imaging (MRI) procedure with respect to the patient. This may also include “landmarking” the patient, which is a process of accurately registering the position of the patient with respect to the coordinate axes of the MRI sensor.

The local technician may then contact the remote technician so as to establish a voice and data connection. Where the term “technician” is used, this is not intended to describe anything but a minimum level of qualification of the person providing the service, and a physician or other trained person may equally perform the service.

Initially a “scout” image of the patient may obtained. A scout image is an image of lower resolution than may be subsequently used for the actual examination, but which may be obtained more rapidly and with less SAR than the full image. The “scout” image is used for examination planning by at least one of the local or remote technician.

The examination may be planned, in whole or in part, by the remote technician, where the higher skill level needed to plan a sophisticated or infrequently performed procedure may be obtained. In a service facility providing remote services, specific individuals may further specialize in particular procedures, so as to provide a high level of skill. Such aspects of the examination as the slices to be acquired, and other examination parameters such as the contrast agent examination times, pulse sequence, EKG gating and synchronization may be planned by the remote technician.

After the examination has been planned, the remote technician may conduct the examination using the local MRI equipment and associated image processing and data processing equipment using the data interface to modify or control the MRI equipment parametric setting and initiate sequences of operations thereof. The remote technician may give voice commands to the patient, such as breathing commands, and adapt the parameters of the examination protocol, if necessary, as the examination proceeds. The examination continues until the remote technician has determined that the remotely controlled or managed portion of examination has been completed.

Equally, after the remote technician has planned the examination and transmitted the parameters for controlling the examination from the remote site to the local site, the technician at the local site may initiate the examination. Control of the examination may be retained at the local site, or returned to the remote technician, depending on the nature of the examination being performed.

At this time, the remote technician may return control of the examination to the local technician and, typically, the connection between the local and remote technicians may be terminated. The examination may be concluded by such actions as removing the IV needle, EKG electrodes, and any other medical apparatus that was connected for the purposes of the examination, removing the patient form the examination table, and returning the patient to another portion of the facility.

Contemporaneously with the ending of the examination, the images and other data are available to a physician so that the images may be read and interpreted and a report prepared. As is known in the art, such reading and interpretation of images and writing of reports may be performed locally or at a remote site, and such outsourcing of image analysis is a common practice. The data images for remote reading and interpretation may be transmitted by a digital representation of the image data over a data network such as the Internet, a virtual private network (VPN), or other communications medium, and or using a communications medium and protocol having a similar function and effect. The report may be returned to the requester by a similar means.

By using a remote technician to plan and perform procedures, and an outsourced image interpretation service, the level of skill needed at the local facility may be reduced. This may be helpful in the nighttime and on weekends, or other times when staffing problems are acute. Alternatively, the method may be used to generally reduce the level of skill required of the technician at the local site by using the technician at the remote site to provide the planning of the procedure. The procedure may then be performed by either the local technician, the remote technician, or the local technician while being monitored or supervised by the remote technician. In this manner a more highly qualified technician may provide service to a number of local sites, each having one or more imaging modalities.

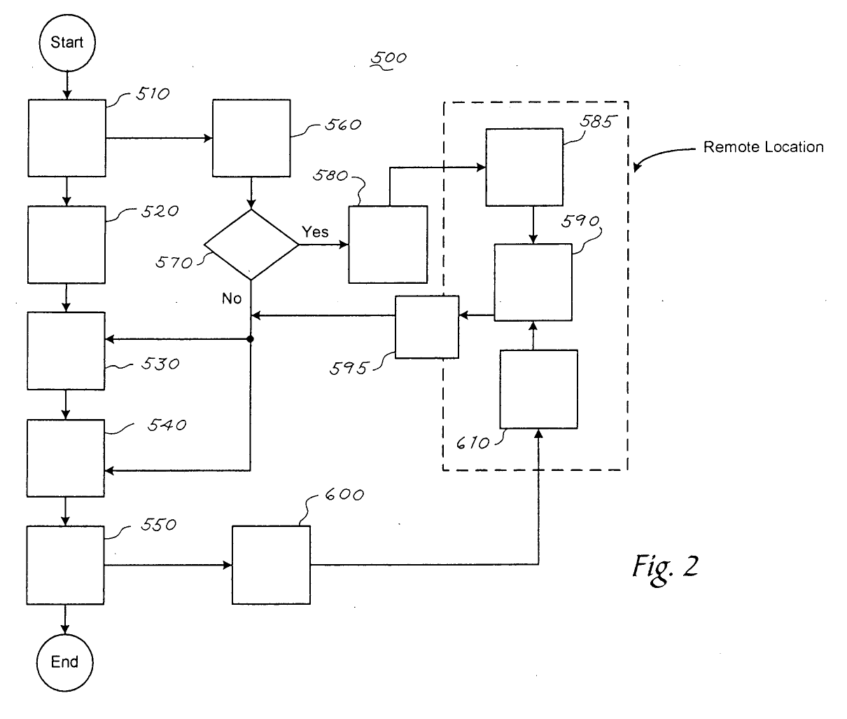

FIG. 2 illustrates the steps in an example 500 of the method. A physician order for a patient examination (step 510) is received, and the patient is brought to the examination suite (step 520). The order may specify, either explicitly or implicitly, the level of skill required by the local technician to perform the set-up, and to conduct the test. This skill level is compared with available personnel for the time period when the imaging examination is to be performed (step 560). If the skill level is not locally available, a decision is taken to secure the services of a remote technician (step 570). It the decision is taken that the remote technician is not needed (NO in step 570), planning and conduct of the examination proceeds locally. The patient is prepared for the examination (step 530), which may include inserting an IV feed and connecting the feed to a contrast agent supply, positioning the patient with respect to the imaging modality, and placing such ancillary equipment as sensing coils and patient sensors such as an EKG. The parameters needed to characterize the particular examination are selected by the local technician and entered in a control interface for the imaging modality (step 540). The imaging modality is used to perform the examination and to obtain and process data for the images (step 550). The examination is then ended, and the image data either locally evaluated or outsourced. The patient is then returned to another area.

When the decision is taken to use the services of a remote technician (YES in step 570), the remote technician is contacted (step 580) and the information in the patient examination order received in step 510 is transmitted to the remote location. At the remote location, the remote technician evaluates the examination order (step 585), the specific imaging modality to be used, and the qualifications of the local technician. The remote technician plans the examination (step 590) and transmits the data needed to establish the examination parameters to the local site (step 595). The data may also include selection of receiving coils, positioning information for the patient, selection of contrast agent, type of patient monitoring, and the like. The data may be communicated in audio or digital form, depending on the nature of the information, and the need for interacting with the local personnel or the patient. The data is input to step 540 for configuring the imaging modality and may be used also in step 530 for preparing the patient. Using the input data, the imaging modality is used to perform the examination and obtain imaging data. At least a portion of the imaging data is transmitted to the remote technician (step 600). The remote technician may evaluate the quality of the image data, or use the image data as a scout image, and determine that a change in one or more parameters of the imaging modality configuration is required (step 610). If such a change is needed, the information is sent to the local site (repeating step 595), or the procedure is considered completed, and an end of procedure message is sent (step 610, not shown).

Where a remote site is used to provide the service described herein, the local site may provide a password or other protocol so that the remote site may connect with the computing equipment at the local site, while respecting computer security and patient data confidentiality. The level of access to patient data may be controlled by the local site, and the level of access may depend on the business relationship of the organization controlling the remote site and the organization controlling the local site.

In another aspect, the method may use a service provider, which may be a separate entity than the local site, to receive requests for planning of imaging studies from one or more local sites, to plan the imaging studies based on the requirements of the local sites for each patient diagnostic exam, and to provide to the local site a plan for the use of the imaging modality in performing the examination, and the appropriate parametric values. The service provider may employ a technician or other professional person with the qualifications appropriate for the type of procedure to be performed. The service provider may sell this service on a per examination basis, or a time of engagement basis, or the like, to the local site. An invoice may be sent from the service provider to the entity responsible for the local site representing an accounting for the cost of the service.

Each patient examination may be considered as a separate instance, and a bill or invoice for the service prepared by the remote site and sent to the local site for each examination or procedure performed. Alternatively, the cost for service may be based on the total amount of service being provided during a billing period, which may be, for example. This may result in a “bulk rate” or discount from the single examination rate.

The service provider may be in contact with one or more local sites having imaging modalities and requirements for planning, supervision, or other examination procedure support using a data communications network such as the Internet, and may also use an audio link to supplement the data communications, where the audio link may be a cellular or conventional telephone circuit, VoIP, or other to be developed communications method, which may combine voice and data.

Large medical facilities may operate in a manner similar to a service provider, where the remote site is controlled by the entity controlling the local site, and the remote site providing the service to one or more local sites, each of which may have more than one imaging modality. The cost for the service provided by the local site may be billed as a separate component of the examination cost, or be included in the examination cost, depending on the reimbursement mode being used in the particular case, or under the regulatory or insurance scheme for the area.

The examples of diseases, syndromes, conditions, and the like, and the types of examination and treatment protocols described herein are by way of example, and are not meant to suggest that the method and apparatus is limited to those named, or the equivalents thereof. As the medical arts are continually advancing, the use of the methods and apparatus described herein may be expected to encompass a broader scope in the diagnosis and treatment of patients.

While the methods disclosed herein have been described and shown with reference to particular steps performed in a particular order, it will be understood that these steps may be combined, sub-divided, or reordered to from an equivalent method without departing from the teachings of the present invention. Accordingly, unless specifically indicated herein, the order and grouping of steps is not a limitation of the present invention.

It is therefore intended that the foregoing detailed description be regarded as illustrative rather than limiting, and that it be understood that it is the following claims, including all equivalents, that are intended to define the spirit and scope of this invention.

What is claimed is:

1. A method of purchasing support for an examination procedure, the method comprising:
providing an imaging modality at a first site;
providing a communications interface between the imaging modality and a communications network;
transmitting data through the communications interface, the data representing requirements for the examination procedure;
receiving data through the communications interface, the data suitable for establishing parametric values of settings of the imaging modality for conducting the examination procedure, and setting the parameters of the imaging modality based on the received data.

2. The method of claim 1, further comprising the step of transmitting a log-in password, the log-in password permitting communications to be received through the communications interface from a second site.

3. The method of claim 3, wherein the first site and the second site are located in separate buildings.

4. The method of claim 1, further comprising the step of receiving a command over the communications interface, the command initiating the examination.

5. The method of claim 1, further comprising the step of transmitting data obtained by the imaging modality through the communications interface.

6. The method of claim 1, further comprising receiving data through the communications interface representing a change of one or more of the parametric settings of the imaging modality.

7. The method of claim 1, wherein the imaging modality is a magnetic resonance imaging (MRI) device.

8. The method of claim 1, further comprising the step of receiving an invoice for the cost of the examination support provided to the first site.

9. The method of claim 1, wherein the data is transmitted by modulation of the data on a carrier wave.

10. The method of claim 1, wherein the communications network is the Internet.

11. A method of selling support for an examination procedure, the method comprising:
providing a computer, a image display, and a computer interface at a first site;
providing a communications interface to a communications network;
receiving data through the communications interface representing the requirements for the examination;
determining parametric values of settings for an imaging modality based on the requirements;
transmitting data representing parametric values of settings through the communications interface.

12. The method of claim 11, further comprising the step of sending a command through the communications interface to initiate the examination procedure.

13. The method of claim 11, further comprising the step of sending data through the communications interface, the data representing a change of one or more of the parametric settings of the imaging modality.

14. The method of claim 11, wherein the imaging modality is a magnetic resonance imaging (MRI) device.

15. The method of claim 11, further comprising the step of sending an invoice for the cost of the examination support.

16. The method of claim 11, wherein the data is transmitted by modulation on a carrier wave.

17. The method of claim 1, wherein the communications network is the Internet.

18. A method of medical imaging, the method comprising:
providing an imaging modality;
interfacing the imaging modality to a communications interface having an interface to a communications network; and
sending information including image data and values of parametric settings of the imaging modality to the communications interface for transmission on the communications network;
receiving information from the communications interface causing a change to a value of one or more of the parametric settings of the imaging modality.

19. The method of claim 18, wherein the imaging modality is a magnetic resonance imaging (MRI) device.

20. The method of claim 18, wherein the parametric settings include at least one of pulse sequence specification, respiratory gating data, slice thickness, or electrocardiogram gating.

21. The method of claim 18, wherein the information received includes a command for the administration of a contrast agent.

22. The method of claim 18, further comprising sending image data produced by the imaging modality to another location for interpretation.

23. The method of claim 22, further comprising receiving the interpretation of the image data from the another location.

24. A method of remotely performing medical imaging, the method comprising:
providing a computer configured to accept data including medical image data and imaging modality parametric control data received by a network communications interface;
displaying at least one of a medical image or imaging modality parametric control data on a image display unit; and
changing a value of at least one of the imaging modality parametric control data, and transmitting the data including at least the changed control data using the network communications interface.

25. The method of claim 24, wherein the transmitted data includes a command for the administration of a contrast agent.

26. A computer-readable medium, the contents of which enable a computer system to perform a method of accepting remote control of an imaging modality, the method comprising:
storing information characterizing the parameters of operation of an imaging modality;
transmitting the parameters of operation to a network interface so as to be communicated to a remote location;
receiving data from the network interface resulting in a change in a value of at least one of the parameters of operation; and
controlling the operation of the imaging modality in accordance with at least the changed value;

27. The computer-readable medium of claim 26, wherein the method further comprises transmitting image data obtained by the imaging modality to the network interface so as to be communicated to the remote location.

28. The computer-readable medium of claim 26, wherein the data is communicated over the Internet.

29. A computer-readable medium, the contents of which enable a computer system to perform a method of remote control of an imaging modality, the method comprising:
receiving information characterizing examination requirements over a network interface;
modifying a value of at least one parameter of operation of an imaging modality; and
transmitting the modified value over a network interface to a remote location having the imaging modality.

30. The computer-readable medium of claim 20, further comprising the step of receiving data obtained by the imaging modality based on the at least one modified value.
